(19) United States
(12) Patent Application Publication
(10) Pub. No.: US 2024/0285165 A1
(43) Pub. Date: Aug. 29, 2024

(54) COMMUNICATION ADAPTER AND METHOD FOR TRANSFERRING DATA

(71) Applicant: BIOTRONIKSE & Co. KG, Berlin (DE)

(72) Inventor: Thomas DOERR, Berlin (DE)

(73) Assignee: BIOTRONIKSE & Co. KG, Berlin (DE)

(21) Appl. No.: 18/560,966

(22) PCT Filed: Jun. 20, 2022
(86) PCT No.: PCT/EP2022/066676

§ 371 Date: Nov. 15, 2023

(30) Foreign Application Priority Data
Jul. 16, 2021 (EP) 21185990.5

(51) Int. Cl. A61B 5/00; G16H 40/67

(52) U.S. Cl. CPC A61B 5/0031; G16H 40/67

(57) ABSTRACT

The invention relates to a communication adapter and a computer implemented method for use with an implantable medical device, in particular a pacemaker, a defibrillator and/or a neuro-stimulator, for transferring data between the implantable medical device and a mobile device, in particular a smartphone or tablet computer. In addition, the invention relates to protective case and a communication adapter system comprising the communication adapter.

### CROSS-REFERENCE TO RELATED APPLICATIONS

This application is the United States National Phase under 35 U.S.C. § 371 of PCT International Patent Application No. PCT/EP2022/066676, filed on Jun. 20, 2022, which claims the benefit of European Patent Application No. 21185990.5, filed on Jul. 16, 2021, the disclosures of which are hereby incorporated by reference herein in their entireties.

### TECHNICAL FIELD

The present invention relates to a communication adapter for use with an implantable medical device, in particular a pacemaker, a defibrillator and/or a neuro-stimulator, for transferring data between the implantable medical device and a mobile device, in particular a smartphone or tablet computer.

Furthermore, the present invention relates to a communication adapter system and a protective case each comprising the communication adapter.

In addition, the present invention relates to a computer implemented method for transferring data between an implantable medical device, in particular a pacemaker, a defibrillator and/or a neuro-stimulator, and a mobile device, in particular a smartphone or tablet computer, by means of a communication adapter.

### BACKGROUND

European Publication No. 1762955 A1 discloses a communication adapter for use with a portable ambulatory medical or therapeutic device, in particular a device for the diagnosis or treatment of a glucose metabolism disorder, for transferring data between the medical or therapeutic device and a computer for displaying operating parameters or measurement data of the device and/or for operating the device, wherein the medical or therapeutic device comprises a device processor for controlling the device and a device adapter interface for communication of the device processor with the communication adapter, and wherein the communication adapter comprises an adapter processor for controlling the communication adapter, an adapter device interface for communication of the communication adapter with the device, an adapter computer interface for communication of the adapter processor with a computer interface of the computer and a device driver with associated transmission protocol.

Implant systems that are able to communicate with a mobile device such as a smartphone are usually equipped with a Bluetooth Low Energy interface.

Disadvantages of a Bluetooth Low Energy interface in implants are, on the one hand, an increased power consumption of this telemetry function especially if the connection is established frequently and, on the other hand, the changing standardizations and possible discontinuation of the Bluetooth Low Energy transmission protocol on the smartphone side over time.

There is thus a risk that no suitable smartphone that can communicate with the Bluetooth Low Energy interface of the implant will be available throughout the operating life of the implant which is typically 15 years.

The present disclosure is directed toward overcoming one or more of the above-mentioned problems, though not necessarily limited to embodiments that do.

### SUMMARY

It is therefore an object of the present invention to provide an improved communication adapter for use with an implantable medical device that offers low energy consumption and that can communicate with the interface of the implant throughout its operating life.

At least the object is solved by a communication adapter for use with an implantable medical device, in particular a pacemaker, a defibrillator and/or a neuro-stimulator, for transferring data between the implantable medical device and a mobile device, in particular a smartphone or tablet computer having the features of claim 1.

At least the object is furthermore solved by a communication adapter system having the features of claim 11 and a protective case having the features of claim 12.

In addition, at least the object is solved by a computer implemented method for transferring data between an implantable medical device, in particular a pacemaker, a defibrillator and/or a neuro-stimulator, and a mobile device, in particular a smartphone or tablet computer, by means of a communication adapter having the features of claim 13.

Moreover, at least the object is solved by a computer program having the features of claim 14 and a computer-readable data carrier having the features of claim 15.

Further developments and advantageous embodiments are defined in the dependent claims.

The present invention provides a communication adapter for use with an implantable medical device, in particular a pacemaker, a defibrillator and/or a neuro-stimulator, for transferring data between the implantable medical device and a mobile device, in particular a smartphone or tablet.

Said communication adapter comprises a MICS telemetry interface for data transfer between the communication adapter and the implantable medical device.

Moreover, said communication adapter comprises a mobile device wireless transmission interface, in particular a Bluetooth LE interface (Bluetooth Low Energy interface) for data transfer between the communication adapter and the mobile device, and an energy source, in particular a battery, configured to power the communication adapter, wherein the communication adapter is configured to transfer data sent via the MICS telemetry interface by the implantable medical device to the mobile device via the mobile device wireless transmission interface and to transfer data sent via the mobile device wireless transmission interface by the mobile device to the implantable medical device via the MICS telemetry interface.

Furthermore, the present invention provides a communication adapter system comprising an adhesive film attachable to the mobile device and the communication adapter according to the present invention, comprising or connected to the first MICS-band antenna, the second MICS-band antenna and the Bluetooth-band antenna, wherein the first MICS-band antenna, the second MICS-band antenna and the Bluetooth-band antenna are arranged on a surface of the adhesive film. The communication adapter is advantageously formed so thin that it can be inserted behind a standard smartphone case.

In addition, the present invention provides a protective case, in particular a Qi-compatible battery case, for a mobile device comprising a frame mountable around an edge of the mobile device, in particular a smartphone or tablet computer; and the communication adapter.

Moreover, the present invention provides a computer implemented method for transferring data between an implantable medical device, in particular a pacemaker, a defibrillator and/or a neuro-stimulator, and a mobile device, in particular a smartphone or tablet computer, by means of a communication adapter.

The method comprises providing a MICS telemetry interface for data transfer between the communication adapter and the implantable medical device.

The method further comprises providing a mobile device wireless transmission interface, in particular a Bluetooth LE interface, for data transfer between the communication adapter and the mobile device, providing an energy source, in particular a battery, configured to power the communication adapter, wherein the communication adapter transfers data sent via the MICS telemetry interface by the implantable medical device to the mobile device via the mobile device wireless transmission interface and transfers data sent via the mobile device wireless transmission interface by the mobile device to the implantable medical device via the MICS telemetry interface.

An idea of the present invention is to provide a communication adapter that converts a communication standard of a mobile device, i.e., Bluetooth low energy, to a communication of an implantable medical device, namely a MICS band telemetry. Using MICS band telemetry, the implantable medical device can thus communicate easily and inexpensively with a mobile device such as a smartphone throughout the operating life of the implant which is typically 15 years. Any potential issues due to changing standardizations and/or compatibility issues because of updates of the Bluetooth Low Energy transmission protocol on the smartphone side over time thus do not affect the implantable medical device which uses solely MICS band telemetry.

An example of a purely therapeutic implant/implantable medical device is, e.g., a stimulator/electrode for deep brain stimulation (e.g., Parkinson's therapy or therapy of depression). The therapy consists of the delivery of pulse trains without collecting diagnostic data from the stimulator.

An example of a purely diagnostic implant is, e.g., a cardiac rhythm monitor. The diagnostic function consists of continuous recording of the patient's ECG and automatic evaluation of abnormalities of the heart rhythm. If such are detected, an ECG recording is stored and typically automatically transmitted to a remote monitoring system.

An example of an implant with therapeutic and diagnostic functions is, e.g., a cardiac pacemaker. The pacemaker is typically implanted subcutaneously in the upper right thoracic region and the electrode is placed in the patient's heart via a large vein. The therapeutic function consists of delivering stimulation pulses to trigger a cardiac action, provided there is no spontaneous cardiac action in the patient. The diagnostic function consists, for example, in the continuous recording of the patient's ECG and automatic evaluation of abnormalities of the heart rhythm. If such are detected, an ECG recording is stored and typically automatically transmitted to a remote monitoring system.

According to an aspect of the present invention, the communication adapter is configured to use a first encryption and/or authentication method for MICS band communication and a second encryption and/or authentication method for mobile device wireless communication. Thus, an authenticated and secure communication between the mobile device and the implantable medical device can be provided.

According to a further aspect of the present invention, the MICS telemetry interface comprises a MICS-band-radio module comprising an encryption unit, in particular a hardware- and/or software-based encryption unit, configured to encrypt a communication between the MICS telemetry interface and the implantable medical device. The communication between the mobile device and the implantable medical device can thus be rendered more secure.

According to a further aspect of the present invention, the communication adapter comprises an authentication unit, in particular a hardware- and/or software-based authentication unit, configured to authenticate a user of the mobile device in order to access patient-related data stored in a data storage unit of the communication adapter and/or the implantable medical device. By authenticating the user of the mobile device, access to the implantable medical device can be restricted to only authorized users. This provides an additional layer of security.

According to a further aspect of the present invention, the communication adapter comprises or is connected to at least a first MICS-band antenna, a second MICS-band antenna and a Bluetooth-band antenna. This provides the advantage of antenna diversity.

According to a further aspect of the present invention, the communication adapter is integratable into a protective case of the mobile device. This way, no additional space is needed in order to accommodate the communication adapter.

According to a further aspect of the present invention, the communication adapter is configured to be supplied with power by means of a reverse wireless charging function of the mobile device, in particular in accordance to the Qi standard. The communication adapter can thus be advantageously provided with energy by means of reverse wireless charging. Therefore, there is no need for a cord-based power supply.

According to a further aspect of the present invention, the communication adapter comprises a non-volatile buffer configured to at least temporarily store information and transfer it to the implantable medical device and/or the mobile device at a later time. The communication adapter thus does not have to be connected to the implant and the mobile device at the same time for its function, i.e., it contains a non-volatile buffer to temporarily store information and forward it at a later time.

According to a further aspect of the present invention, the communication adapter is adapted such that the energy source is replaceable or rechargeable. This enables ease of use and facilitates replacement of the energy source when necessary.

According to a further aspect of the present invention, the communication adapter is configured to support multi-channel ECG data transmission having a latency of less than 1 second, in particular less than 0.1 seconds, to the mobile device, and wherein a maximum data rate for MICS communication is at least 190 kbit/s. This advantageously enables an efficient and an effective data transfer between the implantable medical device and the mobile device.

The herein described features of the communication adapter for use with an implantable medical device are also disclosed for the computer implemented method for transferring data between an implantable medical device and a mobile device and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a schematic view of a communication adapter for use with an implantable medical device according to a first and second embodiment of the present invention;

FIG. 2 shows a schematic view of a communication adapter for use with an implantable medical device according to the first embodiment of the present invention;

FIG. 3 shows a schematic view of a communication adapter implemented as a key fob for use with the implantable medical device according to the second embodiment of the present invention; and

FIG. 4 shows a flowchart of a computer implemented method for transferring data between an implantable medical device and a mobile device according to the first and second embodiment of the present invention.

### DETAILED DESCRIPTION

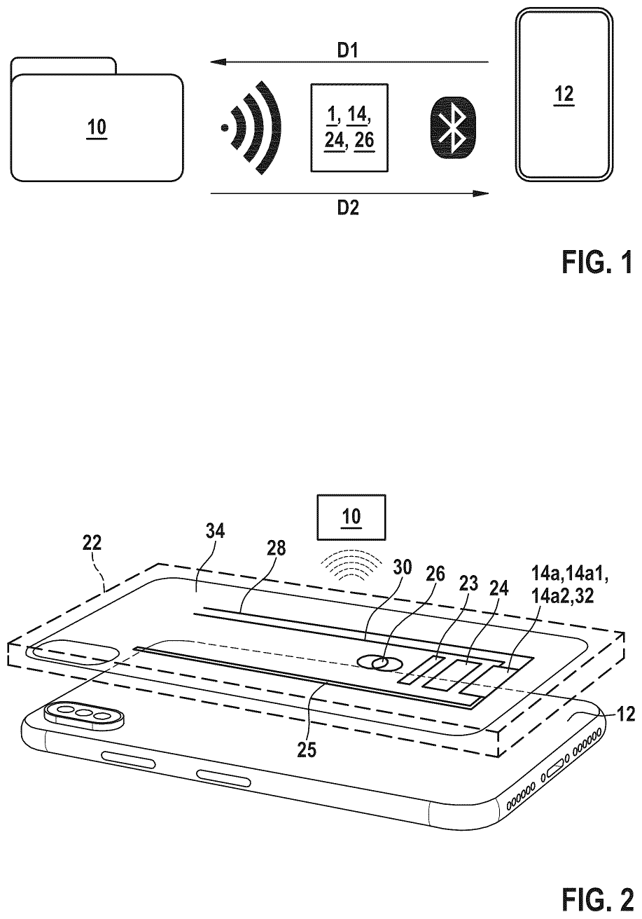

The communication adapter 1 of FIG. 1 for use with an implantable medical device 10, in particular a pacemaker, a defibrillator and/or a neuro-stimulator, for transferring data DI, D2 between the implantable medical device 10 and a mobile device 12, in particular a smartphone or tablet, comprises a MICS telemetry interface 14 for data transfer between the communication adapter 1 and the implantable medical device 10.

The communication adapter 1 further comprises a mobile device wireless transmission interface 24, in particular a Bluetooth LE interface for data transfer between the communication adapter 1 and the mobile device 12. Alternatively, any other suitable wireless transmission protocol such as Wi-Fi, Bluetooth or any other transmission protocol supported by a mobile device can be used. The communication adapter 1 is further configured to be powered by an energy source 26, in particular a battery or an inductive energy source.

The communication adapter 1 is configured to transfer data D1 sent via the MICS telemetry interface 14 by the implantable medical device 10 to the mobile device 12 via the mobile device wireless transmission interface 24 and to transfer data D2 sent via the mobile device wireless transmission interface 24 by the mobile device 12 to the implantable medical device via the MICS telemetry interface 14.

In addition, the communication adapter 1 is configured to use a first encryption and/or authentication method for MICS band communication and a second encryption and/or authentication method for mobile device wireless communication.

FIG. 2 shows a schematic view of a communication adapter for use with an implantable medical device according to the first embodiment of the present invention.

The MICS telemetry interface 14 comprises a MICS-band-radio module 14a comprising an encryption unit 14a1, in particular a hardware-and/or software-based encryption unit 14a1, configured to encrypt a communication between the MICS telemetry interface 14 and the implantable medical device 10.

The communication adapter 1 comprises an authentication unit 14a2, in particular a hardware-and/or software-based authentication unit 14a2, configured to authenticate a user of the mobile device 12 in order to access patient-related data stored in a data storage unit 32 of the communication adapter 1 and/or the implantable medical device 10.

Furthermore, the communication adapter 1 comprises or is connected to at least a first MICS-band antenna 28, a second MICS-band antenna 30 and a Bluetooth-band antenna 25.

The communication adapter 1 is configured to be supplied with power by means of a reverse wireless charging function of the mobile device 12, in particular in accordance to the Qi standard.

Moreover, the communication adapter 1 comprises a non-volatile buffer 23 configured to at least temporarily store information and transfer it to the implantable medical device 10 and/or the mobile device 12 at a later time.

The communication adapter 1 is configured to support multi-channel ECG data transmission having a latency of less than 1 second, in particular less than 0.1 seconds, to the mobile device 12, and wherein a maximum data rate for MICS communication is at least 190 kbit/s. The communication adapter 1 further supports an extremely low power method of establishing MICS communication known as Search Block Trigger. Its power consumption is less than 2% of a usable power of the implantable medical device to establish communication.

The communication adapter 1 is further configured to support use cases in a 5G connection of the connected mobile device enabled by the low latency of 5G technology such as remote programming of the implant with a programmer operated in the cloud. The communication adapter 1 is connected to the implantable medical device 10 and the mobile device 12 simultaneously for its function.

A Communication adapter system shown in FIG. 2 comprises an adhesive film 34 attachable to the mobile device 12 and the communication adapter 1, comprising or connected to the first MICS-band antenna 28, the second MICS-band antenna 30 and the Bluetooth-band antenna 25, wherein the first MICS-band antenna 28, the second MICS-band antenna 30 and the Bluetooth-band antenna 25 are arranged on a surface of the adhesive film 34.

The communication adapter 1 is further configured to be powered by the energy source 26, which according to the first embodiment is an inductive energy source given by an inductive coil of the mobile device 12.

The communication adapter 1 is furthermore integratable into a protective case 22 of the mobile device 12. The protective case 22, in particular a Qi-compatible battery case, for a mobile device 12 comprises a frame mountable around an edge of the mobile device 12, in particular a smartphone or tablet computer; and the communication adapter 1. Alternatively, the communication adapter 1 may be integrated into a USB-cable.

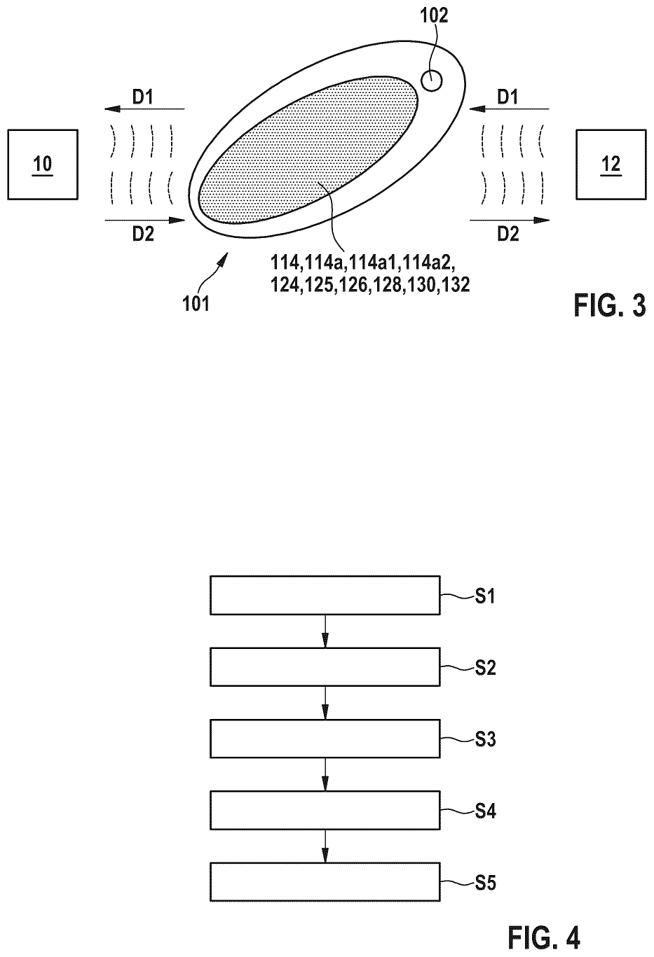

FIG. 3 shows a schematic view of a communication adapter 101 being implemented as a key fob for use with the implantable medical device according to a fourth embodiment of the present invention. The communication adapter 101 comprises an opening 102 through which a key ring or holder can be inserted.

The communication adapter 101 is capable of transferring data D1, D2 between the implantable medical device 10 and a mobile device 12, in particular a smartphone or tablet.

The communication adapter 101 comprises a MICS telemetry interface 114 for data transfer between the communication adapter 101 and the implantable medical device 10. The communication adapter 101 further comprises a mobile device wireless transmission interface 124 for data transfer between the communication adapter 101 and the mobile device 12 and an energy source 126, in particular a battery, configured to power the communication adapter 101. The communication adapter 101 is adapted such that the energy source 126 is replaceable or rechargeable.

The communication adapter 101 is configured to transfer data D1 sent via the MICS telemetry interface 114 by the implantable medical device 10 to the mobile device 12 via the mobile device wireless transmission interface 124 and to transfer data D2 sent via the mobile device wireless transmission interface 124 by the mobile device 12 to the implantable medical device 10 via the MICS telemetry interface 114.

The MICS telemetry interface 114 comprises a MICS-band-radio module 114a comprising an encryption unit 114a1, in particular a hardware-and/or software-based encryption unit 114a1, configured to encrypt a communication between the MICS telemetry interface 114 and the implantable medical device 10.

The communication adapter 101 comprises or is connected to at least a first MICS-band antenna 128, a second MICS-band antenna 130 and a Bluetooth-band antenna 125.

The communication adapter 101 comprises an authentication unit 114a2, in particular a hardware-and/or software-based authentication unit 114a2, configured to authenticate a user of the mobile device 12 in order to access patient-related data stored in a data storage unit 132 of the communication adapter 101 and/or the implantable medical device 10.

FIG. 4 shows a flowchart of a computer implemented method for transferring data D1, D2 between an implantable medical device 10, in particular a pacemaker, a defibrillator and/or a neuro-stimulator, and a mobile device 12, in particular a smartphone or tablet computer, by means of a communication adapter 1.

The method comprises providing S1 a MICS telemetry interface 14 for data transfer between the communication adapter 1 and the implantable medical device 10 and providing S2 a mobile device wireless transmission interface 24 for data transfer between the communication adapter 1 and the mobile device 12.

Furthermore, the method comprises providing S3 an energy source 26, in particular a battery or an inductive energy source, configured to power the communication adapter 1. The communication adapter 1 transfers S4 data D1 sent via the MICS telemetry interface 14 by the implantable medical device 10 to the mobile device 12 via the mobile device wireless transmission interface 24 and transfers S5 data D2 sent via the mobile device wireless transmission interface 24 by the mobile device 12 to the implantable medical device 10 via the MICS telemetry interface 14.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### Reference Signs

1, 101 communication adapter
10 implantable medical device
12 mobile device
14, 114 MICS telemetry interface
14a, 114a MICS-band-radio module
14a1, 114a1 encryption unit
14a2, 114a2 authentication unit
22 protective case
23 non-volatile buffer
24, 124 mobile device wireless transmission interface
25, 125 Bluetooth-band antenna
26, 126 energy source
28, 128 first MICS-band antenna
30, 130 second MICS-band antenna
32, 132 data storage unit
34 adhesive film
102 opening
D1, D2 data
S1-S5 method steps

1. Communication adapter for use with an implantable medical device, in particular a pacemaker, a defibrillator and/or a neuro-stimulator, for transferring data between the implantable medical device and a mobile device, in particular a smartphone or tablet, comprising:
a MICS telemetry interface for data transfer between the communication adapter and the implantable medical device; and
a mobile device wireless transmission interface, in particular a Bluetooth LE interface, for data transfer between the communication adapter and the mobile device, wherein the communication adapter is configured to be powered by an energy source, in particular a battery or an inductive energy source, and wherein the communication adapter is configured to transfer data sent via the MICS telemetry interface by the implantable medical device to the mobile device via the mobile device wireless transmission interface and to transfer data sent via the mobile device wireless transmission interface by the mobile device to the implantable medical device via the MICS telemetry interface.

2. Communication adapter of claim 1, wherein the communication adapter is configured to use a first encryption and/or authentication method for MICS band communication and a second encryption and/or authentication method for mobile device wireless communication.

3. Communication adapter of claim 1, wherein the MICS telemetry interface comprises a MICS-band-radio module comprising an encryption unit, in particular a hardware- and/or software-based encryption unit, configured to encrypt a communication between the MICS telemetry interface and the implantable medical device.

4. Communication adapter of claim 1, wherein the communication adapter comprises an authentication unit, in particular a hardware- and/or software-based authentication unit, configured to authenticate a user of the mobile device in order to access patient-related data stored in a data storage unit of the communication adapter and/or the implantable medical device.

5. Communication adapter of claim 1, wherein the communication adapter comprises or is connected to at least a first MICS-band antenna, a second MICS-band antenna and a Bluetooth-band antenna.

6. Communication adapter of claim 1, wherein the communication adapter is integratable into a protective case of the mobile device.

7. Communication adapter of claim 1, wherein the communication adapter is configured to be supplied with power by means of a reverse wireless charging function of the mobile device, in particular in accordance to the Qi standard.

8. Communication adapter of claim 1, wherein the communication adapter comprises a non-volatile buffer configured to at least temporarily store information and transfer it to the implantable medical device and/or the mobile device at a later time.

9. Communication adapter of claim 1, wherein the communication adapter is adapted such that the energy source is replaceable or rechargeable.

10. Communication adapter of claim 1, wherein the communication adapter is configured to support multi-channel ECG data transmission having a latency of less than 1 second, in particular less than 0.1 seconds, to the mobile device, and wherein a maximum data rate for MICS communication is at least 190 kbit/s.

11. Communication adapter system comprising an adhesive film attachable to the mobile device and the communication adapter of claim 1, comprising or connected to the first MICS-band antenna, the second MICS-band antenna and the Bluetooth-band antenna, wherein the first MICS-band antenna, the second MICS-band antenna and the Bluetooth-band antenna are arranged on a surface of the adhesive film.

12. Protective case, in particular a Qi-compatible battery case, for a mobile device comprising a frame mountable around an edge of the mobile device, in particular a smartphone or tablet computer; and the communication adapter of claim 1.

13. Computer implemented method for transferring data between an implantable medical device, in particular a pacemaker, a defibrillator and/or a neuro-stimulator, and a mobile device, in particular a smartphone or tablet computer, by means of a communication adapter, comprising the steps of:
providing a MICS telemetry interface for data transfer between the communication adapter and the implantable medical device;
providing a mobile device wireless transmission interface, in particular a Bluetooth LE interface for data transfer between the communication adapter and the mobile device; and
providing an energy source, in particular a battery or an inductive energy source, configured to power the communication adapter, wherein the communication adapter transfers data sent via the MICS telemetry interface by the implantable medical device to the mobile device via the mobile device wireless transmission interface and transfers data sent via the mobile device wireless transmission interface by the mobile device to the implantable medical device via the MICS telemetry interface.

14. Computer program with program code to perform the method of claim 13 when the computer program is executed on a computer.

15. Computer-readable data carrier containing program code of a computer program for performing the method of claim 13 when the computer program is executed on a computer.
